SEQUENCE LISTING (34 sequences)

SEQ ID NO: 1 - protein, 8 aa, Artificial Sequence
  Synthetic peptide
QHTQITKV

SEQ ID NO: 2 - protein, 8 aa, Artificial Sequence
  Synthetic peptide
QHSQITKV

SEQ ID NO: 3 - protein, 8 aa, Artificial Sequence
  Synthetic peptide
QHXQIXKX

SEQ ID NO: 4 - protein, 13 aa, Artificial Sequence
  Synthetic peptide
PFDEDQHTQITKV

SEQ ID NO: 5 - protein, 12 aa, Artificial Sequence
  Synthetic peptide
FDEDQHTQITKV

SEQ ID NO: 6 - protein, 11 aa, Artificial Sequence
  Synthetic peptide
DEDQHTQITKV

SEQ ID NO: 7 - protein, 10 aa, Artificial Sequence
  Synthetic peptide
EDQHTQITKV

SEQ ID NO: 8 - protein, 9 aa, Artificial Sequence
  Synthetic peptide
DQHTQITKV

SEQ ID NO: 9 - protein, 8 aa, Artificial Sequence
  Synthetic peptide
QHTQITKL

SEQ ID NO: 10 - protein, 8 aa, Artificial Sequence
  Synthetic peptide
QHTQITKI

SEQ ID NO: 11 - protein, 8 aa, Artificial Sequence
  Synthetic peptide
QHTQISKV

SEQ ID NO: 12 - protein, 8 aa, Artificial Sequence
  Synthetic peptide
QHTQISKL

SEQ ID NO: 13 - protein, 8 aa, Artificial Sequence
  Synthetic peptide
QHTQISKI

SEQ ID NO: 14 - protein, 13 aa, Artificial Sequence
  Synthetic peptide
PFDEDQHSQITKV

SEQ ID NO: 15 - protein, 12 aa, Artificial Sequence
  Synthetic peptide
FDEDQHSQITKV

SEQ ID NO: 16 - protein, 11 aa, Artificial Sequence
  Synthetic peptide
DEDQHSQITKV

SEQ ID NO: 17 - protein, 10 aa, Artificial Sequence
  Synthetic peptide
EDQHSQITKV

SEQ ID NO: 18 - protein, 9 aa, Artificial Sequence
  Synthetic peptide
DQHSQITKV

SEQ ID NO: 19 - protein, 8 aa, Artificial Sequence
  Synthetic peptide
QHSQITKV

SEQ ID NO: 20 - protein, 8 aa, Artificial Sequence
  Synthetic peptide
QHSQITKL

SEQ ID NO: 21 - protein, 8 aa, Artificial Sequence
  Synthetic peptide
QHSQITKI

SEQ ID NO: 22 - protein, 8 aa, Artificial Sequence
  Synthetic peptide
QHSQISKV

SEQ ID NO: 23 - protein, 8 aa, Artificial Sequence
  Synthetic peptide
QHSQISKL

SEQ ID NO: 24 - protein, 8 aa, Artificial Sequence
  Synthetic peptide
QHSQISKI

SEQ ID NO: 25 - protein, 8 aa, Artificial Sequence
  Synthetic peptide
QHTQITKV

SEQ ID NO: 26 - DNA, 19 bp, Artificial sequence
  Synthetic oligonucleotide
caggtggtggagcaagatg

SEQ ID NO: 27 - DNA, 27 bp, Artificial sequence
  Synthetic oligonucleotide
ccgagatctctgaagtgaagatggatg

SEQ ID NO: 28 - DNA, 30 bp, Artificial sequence
  Synthetic oligonucleotide
cctctttgtgactatgtggactgatgtcgg

SEQ ID NO: 29 - DNA, 28 bp, Artificial sequence
  Synthetic oligonucleotide
gtggataccccctcccccagcctagacc

SEQ ID NO: 30 - DNA, 24 bp, Artificial sequence
  Synthetic oligonucleotide
gctgaagtggctgaagagctctga

SEQ ID NO: 31 - DNA, 24 bp, Artificial sequence
  Synthetic oligonucleotide
ttgagtgaaactgatgaggtatgg

SEQ ID NO: 32 - protein, 4 aa, Artificial sequence
  Synthetic peptide
ITKV

SEQ ID NO: 33 - protein, 5 aa, Artificial sequence
  Synthetic peptide
QITKV

SEQ ID NO: 34 - protein, 8 aa, Artificial sequence
  Synthetic peptide
SVHTIQKQ

(19) United States
(12) Patent Application Publication
(10) Pub. No.: US 2017/0165317 A1
(43) Pub. Date: Jun. 15, 2017

(54) METHOD FOR MITIGATING OR ALLEVIATING SYNAPTIC AND COGNITIVE DEFICITS

(71) Applicants: Trustees of Dartmouth College, Hanover, NH (US); Universidad Nacional de Education a Distancia, Madrid (ES)

(72) Inventors: Mark Robert SPALLER, Hanover, NH (US); Jose Antonio Esteban GARCIA, Madrid (ES); Cesar VENERO, Madrid (ES); Dina Shira KNAFO, Madrid (ES)

(21) Appl. No.: 15/039,269

(22) PCT Filed: Nov. 11, 2014
(86) PCT No.: PCT/US2014/064929

§ 371 Date: May 25, 2016

Related U.S. Application Data
(60) Provisional application No. 61/908,909, filed on Nov. 26, 2013.

(51) Int. Cl. A61K 38/08; A61K 9/00

(52) U.S. Cl. CPC A61K 38/08; A61K 9/0019; A61K 9/0085

(57) ABSTRACT

A method for mitigating or alleviating synaptic and cognitive deficits associated with a β-amyloidogenic disease using a peptide, or derivative or peptidomimetic thereof, derived from the C-terminus of PTEN is provided, as is a kit containing said peptide.

This application claims benefit of priority of U.S. Provisional Application No. 61/908,909 filed Nov. 26, 2013, the content of which is incorporated herein by reference in its entirety.

### INTRODUCTION

### Background

Gradual accumulation of amyloid-beta (Aβ) peptide induces a series of synaptic and neuronal dysfunctions, which appear to be responsible for cognitive deficits ranging in severity from mild-cognitive impairment (MCI) to Alzheimer's dementia (Terry, et al. (1991) Ann. Neurol. 30:572-580; Selkoe (2002) Science 298:789-791; Jack, et al. (2010) Brain 133:3336-3348; Selkoe (2011) Nat. Med. 17:1060-1065). Accumulating evidence indicates that soluble Aβ assemblies directly alter synaptic plasticity mechanisms by inhibiting long-term potentiation (LTP) and facilitating long-term depression (LTD) in hippocampal neurons (Li, et al. (2009) Neuron 62:788-801; Hsieh, et al. (2006) Neuron 52:831-843; Cisse, et al. (2011) Nature 469:47-52; Shankar, et al. (2008) Nat. Med. 14:837-842; Walsh, et al. (2002) Nature 416:35-539; Cullen, et al (1996) Neuroreport. 8:87-92; Lambert, et al. (1998) Proc. Natl. Acad. Sci. USA 95:6448-6453). Therefore, it appears that Aβ shifts the synaptic plasticity balance toward a pathologically enhanced form of depression. PIP₃ signaling is an important regulator of AMPA receptor (AMPAR) function and synaptic plasticity (Man, et al. (2003) Neuron 38:611-624; Arendt, et al. (2010) Nat. Neurosci. 13:36-44; Peineau, et al. (2007) Neuron 53:703-717), and it has been shown that PI3K (the PIP₃ synthesizing enzyme) favors synaptic potentiation (Arendt, et al. (2010) Nat. Neurosci. 13:36-44), whereas the lipid phosphatase PTEN (down-regulator of PIP₃) mediates depression (Jurado, et al. (2010) EMBO J. 29:2827-2840). In addition, it has been suggested that soluble Aβ oligomers facilitate electrically-evokes LTD in the CA1 region via mGluR or NMDAR activity (Li et al. (2009) Neuron 62:788-801). While US 2005/0282743 teaches that a C-terminal PTEN peptide having the sequence QHTQITKV (SEQ ID NO:1) can be used in targeting a PDZ domain-containing protein in cells, a biological activity associated with this peptide was not described.

### SUMMARY OF THE INVENTION

This invention is a method for mitigating or alleviating synaptic and cognitive deficits associated with a β-amyloidogenic disease by administering an effective amount of a peptide of SEQ ID NO:3 (Gln-His-Xaa₁-Gln-Ile-Xaa₂-Lys-Xaa₃, wherein Xaa₁ and Xaa₂ are independently Ser or Thr, and Xaa₃ is Val, Leu or Ile), or a derivative or peptidomimetic thereof, to a subject in need of treatment. In one embodiment, the β-amyloidogenic disease is Alzheimer's disease. In another embodiment, the peptide, derivative or peptidomimetic is administered to the subject by direct injection, including direct injection into the brain of the subject. In another embodiment, the derivative is N-myristoyl-QHSQITKV (SEQ ID NO:2) or N-myristoyl-QHTQITKV (SEQ ID NO:25). A kit containing an effective amount of a peptide derivative comprising N-myristoyl-QHSQITKV (SEQ ID NO:2) or N-myristoyl-QHTQITKV (SEQ ID NO:25); and a pharmaceutically acceptable carrier is also provided.

### BRIEF DESCRIPTION OF THE DRAWINGS

FIG. 1 shows the results of APP/PS1 and wild-type (WT) littermates (treated with VO-OHpic or with aCSF) tested in the novel object location task. The recognition index was calculated as the time spent exploring the displaced object/time spent exploring both objects. Therefore a score of 0.5 would indicate no preference.

FIG. 2 shows the percentage freezing to the context for vehicle-infused mice and mice infused with VO-OHpic.

FIG. 3 shows the percentage freezing to the tone for vehicle-infused mice and mice infused with VO-OHpic.

FIG. 4 shows the quantification of average fEPSP maximal slops, at 50-60 minutes after induction. P value was determined with Mann-Whitney test.

FIG. 5 shows the results of APP/PS1 and wild-type (WT) littermates treated with PTEN-PDZ peptide tested in the novel object location task. The recognition index was calculated as the time spent exploring the displaced object/time spent exploring both objects.

### DETAILED DESCRIPTION OF THE INVENTION

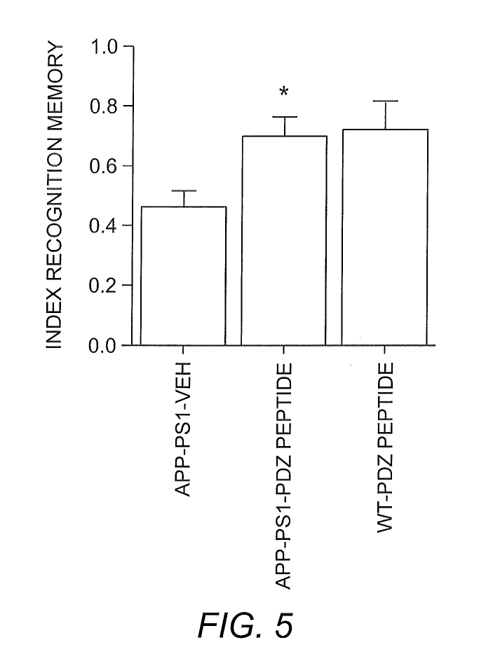

The results presented herein demonstrate the role of PTEN in Aβ-induced synaptic failure and its cognitive consequences. Specifically, PTEN function was manipulated in vivo and in vitro to elucidate and counteract the role of PTEN in the depressing effect of Aβ. It was found that PTEN is a target of Aβ's action, which involves PDZ-dependent recruitment of PTEN into dendritic spines. Moreover, genetic and pharmacological prevention of these PDZ-dependent interactions protects neurons from Aβ-induced toxicity. Specifically, it has now been found that a cell-permeable octapeptide (N-myristoyl-QHSQITKV; SEQ ID NO:2) derived from the C-terminus of PTEN blocks Aβ-induced synaptic depression mediated by PDZ-dependent recruitment of PTEN. In addition, it was found that upon intracerebroventricular injection of this peptide, APP/PS1 mice showed a significant improvement in a spatial learning task (p=0.027, compared to vehicle-infused APP/PS1 mice), to the extent that their recognition index was similar to that of wild-type mice (FIG. 5). These data offer fundamental information about the mechanisms by which Aβ perturbs synaptic function, and reveal PTEN as a critical mediator and therapeutic target in Alzheimer's disease and related conditions mediated by Aβ protein.

Accordingly, this invention provides a method for mitigating or alleviating synaptic or cognitive deficits associated with a β-amyloidogenic disease using an inhibitor that blocks Aβ-induced synaptic depression mediated by PDZ-dependent recruitment of PTEN to into dendritic spines. As is known in the art, PTEN protein acts as a phosphatase to dephosphorylate phosphatidylinositol (3,4,5)-trisphosphate (PtdIns (3,4,5)P₃ or PIP₃). The amino acid sequence of PTEN is known in the art and available under GENBANK Accession Nos. NP_000305 (human) and NP_032986 (mouse). The structure of PTEN (Lee, et al. (1999) Cell 99:323-34) reveals that it is composed of a phosphatase domain, and a C2 domain, wherein the phosphatase domain contains the active site and the C2 domain binds the phospholipid membrane.

Because PDZ proteins share overlapping specificities, particular embodiments of this invention embrace an inhibitor that selectively blocks PDZ-dependent recruitment of PTEN into dendritic spines. As used herein, a “selective inhibitor” is any molecular species that inhibits PDZ-dependent recruitment of PTEN into dendritic spines but which fails to inhibit, or inhibits to a substantially lesser degree other PDZ protein interactions. A selective inhibitor of this invention can be identified using any suitable screening assay that monitors PTEN activity or localization. By way of illustration, libraries of agents can be screened for the ability to protect neurons from synaptic depression induced by APP_{swe/lnd} expression. Inhibitors of the present invention can be any molecular species, with particular embodiments embracing peptides or mimetics thereof. In certain embodiments, an inhibitor that selectively blocks PDZ-dependent recruitment of PTEN is a peptide.

As used herein, the term “peptide” denotes an amino acid polymer that is composed of at least two amino acids covalently linked by an amide bond. Peptides of the present invention are desirably 8 to 20 residues in length, or more desirably 8 to 10 residues in length. In certain embodiments, an inhibitor that selectively blocks PDZ-dependent recruitment of PTEN is an 8 to 20 amino acid residue peptide comprising or consisting of the amino acid sequence Gln-His-Xaa₁-Gln-Ile-Xaa₂-Lys-Xaa₃ (SEQ ID NO:3), wherein Xaa₁ and Xaa₂ are independently Ser or Thr, and Xaa₃ is Val, Leu or Ile. In addition to Ser or Thr, Xaa₂ can be any residues in which there is a hydoxy group at the beta position. Similarly, in addition to Val, Leu or Ile, Xaa₃ can be any residue with an aliphatic side chain. In certain embodiments of the present invention, a selective inhibitor of the invention has an amino acid sequence as listed in Table 1.

TABLE 1

| Source | Inhibitor Peptide Sequence | SEQ ID NO: |
|---|---|---|
| Human | PFDEDQHTQITKV | 4 |
| | FDEDQHTQITKV | 5 |
| | DEDQHTQITKV | 6 |
| | EDQHTQITKV | 7 |
| | DQHTQITKV | 8 |
| | QHTQITKV | 1 |
| | QHTQITKL | 9 |
| | QHTQITKI | 10 |
| | QHTQISKV | 11 |
| | QHTQISKL | 12 |
| | QHTQISKI | 13 |
| Mouse | PFDEDQHSQITKV | 14 |
| | FDEDQHSQITKV | 15 |
| | DEDQHSQITKV | 16 |
| | EDQHSQITKV | 17 |
| | DQHSQITKV | 18 |
| | QHSQITKV | 19 |
| | QHSQITKL | 20 |
| | QHSQITKI | 21 |
| | QHSQISKV | 22 |
| | QHSQISKL | 23 |
| | QHSQISKI | 24 |

In specific embodiments, the peptide of the invention has the sequence QHTQITKV (SEQ ID NO:1) or QHSQITKV (SEQ ID NO:19).

In accordance with the present invention, derivatives of the peptides of the invention are also provided. As used herein, a peptide derivative is a molecule which retains the primary amino acids of the peptide, however, the N-terminus, C-terminus, and/or one or more of the side chains of the amino acids therein have been chemically altered or derivatized. Such derivatized peptides include, for example, naturally occurring amino acid derivatives, for example, allo-threonine, 4-hydroxyproline for proline, 5-hydroxylysine for lysine, homoserine for serine, ornithine for lysine, and the like. Other derivatives or modifications include, e.g., a label, such as fluorescein or tetramethylrhodamine; or one or more post-translational modifications such as acetylation, amidation, formylation, hydroxylation, methylation, myristoylation, palmitoylation, stearoylation, phosphorylation, sulfatation, glycosylation, or lipidation. Indeed, certain chemical modifications, in particular N-terminal glycosylation, have been shown to increase the stability of peptides in human serum (Powell et al. (1993) Pharma. Res. 10:1268-1273). Peptide derivatives also include those with increased membrane permeability obtained by N-myristoylation (Brand, et al. (1996) Am. J. Physiol. Cell. Physiol. 270:C1362-C1369). Exemplary peptide derivatives are N-myristoyl-QHSQITKV (SEQ ID NO:2) and N-myristoyl-QHTQITKV (SEQ ID NO:25).

In addition, a peptide derivative of the invention can include a cell-penetrating sequence which facilitates, enhances, or increases the transmembrane transport or intracellular delivery of the peptide into a cell. For example, a variety of proteins, including the HIV-1 Tat transcription factor, Drosophila Antennapedia transcription factor, as well as the herpes simplex virus VP22 protein have been shown to facilitate transport of proteins into the cell (Wadia and Dowdy (2002) Curr. Opin. Biotechnol. 13:52-56). Further, an arginine-rich peptide (Futaki (2002) Int. J. Pharm. 245:1-7), a polylysine peptide containing Tat PTD (Hashida, et al. (2004) Br. J. Cancer 90(6):1252-8), Pep-1 (Deshayes, et al. (2004) Biochemistry 43(6):1449-57) or an HSP70 protein or fragment thereof (WO 00/31113) is suitable for enhancing intracellular delivery of a peptide or peptidomimetic of the invention into the cell.

While a peptide of the invention can be derivatized with one of the above indicated modifications, it is understood that a peptide of this invention may contain more than one of the above described modifications within the same peptide.

As indicated, the present invention also encompasses peptidomimetics of the peptides disclosed herein. Peptidomimetics refer to a synthetic chemical compound which has substantially the same structural and/or functional characteristics of the peptides of the invention. The mimetic can be entirely composed of synthetic, non-natural amino acid analogues, or can be a chimeric molecule including one or more natural peptide amino acids and one or more non-natural amino acid analogs. The mimetic can also incorporate any number of natural amino acid conservative substitutions as long as such substitutions do not destroy the activity of the mimetic. Routine testing can be used to determine whether a mimetic has the requisite activity, e.g., that it can inhibit Aβ-induced synaptic depression mediated by PDZ-dependent recruitment of PTEN. The phrase “substantially the same,” when used in reference to a mimetic or peptidomimetic, means that the mimetic or peptidomimetic has one or more activities or functions of the referenced molecule.

There are advantages for using a mimetic of a given peptide. For example, there are considerable cost savings and improved patient compliance associated with peptidomimetics, since they can be administered orally compared with parenteral administration for peptides. Furthermore, peptidomimetics can be cheaper to produce than peptides.

Thus, peptides described above have utility in the development of such small chemical compounds with similar biological activities and therefore with similar therapeutic utilities. The techniques of developing peptidomimetics are conventional. For example, peptide bonds can be replaced by non-peptide bonds or non-natural amino acids that allow the peptidomimetic to adopt a similar structure, and therefore biological activity, to the original peptide. Further modifications can also be made by replacing chemical groups of the amino acids with other chemical groups of similar structure. The development of peptidomimetics can be aided by determining the tertiary structure of the original peptide by NMR spectroscopy, crystallography and/or computer-aided molecular modeling. These techniques aid in the development of novel compositions of higher potency and/or greater bioavailability and/or greater stability than the original peptide (Dean (1994) BioEssays 16:683-687; Cohen & Shatzmiller (1993) J. Mol. Graph. 11:166-173; Wiley & Rich (1993) Med. Res. Rev. 13:327-384; Moore (1994) Trends Pharmacol. Sci. 15:124-129; Hruby (1993) Biopolymers 33:1073-1082; Bugg, et al. (1993) Sci. Am. 269:92-98). Once a potential peptidomimetic compound is identified, it may be synthesized and assayed using an assay described.

It will be readily apparent to one skilled in the art that a peptidomimetic can be generated from any of the peptides described herein. It will furthermore be apparent that the peptidomimetics of this invention can be further used for the development of even more potent non-peptidic compounds, in addition to their utility as therapeutic compounds.

Peptide mimetic compositions can contain any combination of non-natural structural components, which are typically from three structural groups: residue linkage groups other than the natural amide bond (“peptide bond”) linkages; non-natural residues in place of naturally occurring amino acid residues; residues which induce secondary structural mimicry, i.e., induce or stabilize a secondary structure, e.g., a beta turn, gamma turn, beta sheet, alpha helix conformation, and the like; or other changes which confer resistance to proteolysis. For example, a polypeptide can be characterized as a mimetic when one or more of the residues are joined by chemical means other than an amide bond. Individual peptidomimetic residues can be joined by amide bonds, non-natural and non-amide chemical bonds other chemical bonds or coupling means including, for example, glutaraldehyde, N-hydroxysuccinimide esters, bifunctional maleimides, N,N′-dicyclohexylcarbodiimide (DCC) or N,N′-diisopropyl-carbodiimide (DIC). Linking groups alternative to the amide bond include, for example, ketomethylene (e.g., —C(═O)—CH₂— for —C(═O)—NH—), aminomethylene (CH₂—NH), ethylene, olefin (CH═CH), ether (CH₂—O), thioether (CH₂—S), tetrazole (CN₄—), thiazole, retroamide, thioamide, or ester (see, e.g., Spatola (1983) in Chemistry and Biochemistry of Amino Acids, Peptides and Proteins, 7:267-357, “Peptide and Backbone Modifications,” Marcel Decker, NY).

As discussed, a peptide can be characterized as a mimetic by containing one or more non-natural residues in place of a naturally occurring amino acid residue. Non-natural residues are known in the art. Particular non-limiting examples of non-natural residues useful as mimetics of natural amino acid residues are mimetics of aromatic amino acids include, for example, D- or L-naphylalanine; D- or L-phenylglycine; D- or L-2 thieneylalanine; D- or L-1, -2, 3-, or 4-pyreneylalanine; D- or L-3 thieneylalanine; D- or L-(2-pyridinyl)-alanine; D- or L-(3-pyridinyl)-alanine; D- or L-(2-pyrazinyl)-alanine; D- or L-(4-isopropyl)-phenylglycine; D-(trifluoromethyl)-phenylglycine; D-(trifluoromethyl)-phenylalanine; D-p-fluoro-phenylalanine; D- or L-p-biphenylphenylalanine; D- or L-p-methoxy-biphenyl-phenylalanine; and D- or L-2-indole(alkyl)alanines, where alkyl can be substituted or unsubstituted methyl, ethyl, propyl, hexyl, butyl, pentyl, isopropyl, iso-butyl, sec-isotyl, iso-pentyl, or a non-acidic amino acid. Aromatic rings of a non-natural amino acid that can be used in place a natural aromatic ring include, for example, thiazolyl, thiophenyl, pyrazolyl, benzimidazolyl, naphthyl, furanyl, pyrrolyl, and pyridyl aromatic rings. By way of illustration, Xaa₃ can be α-aminoisobutyric acid (Aib), aminobutyric acid (Abu), 2-aminopentanoic acid (Ape), 2-aminohexanoic acid (Ahx), or tert-leucine (Tle).

Cyclic peptides or cyclized residue side chains also decrease susceptibility of a peptide to proteolysis by exopeptidases or endopeptidases. Thus, certain embodiments embrace a peptidomimetic of the peptides disclosed herein, whereby one or more amino acid residue side chains are cyclized according to conventional methods.

Mimetics of acidic amino acids can be generated by substitution with non-carboxylate amino acids while maintaining a negative charge; (phosphono)alanine; and sulfated threonine. Carboxyl side groups (e.g., aspartyl or glutamyl) can also be selectively modified by reaction with carbodiimides (R′—N—C—N—R′) including, for example, 1-cyclohexyl-3(2-morpholinyl-(4-ethyl) carbodiimide or 1-ethyl-3(4-azonia-4,4-dimetholpentyl) carbodiimide. Aspartyl or glutamyl groups can also be converted to asparaginyl and glutaminyl groups by reaction with ammonium ions.

Lysine mimetics can be generated (and amino terminal residues can be altered) by reacting lysinyl with succinic or other carboxylic acid anhydrides. Lysine and other alpha-amino-containing residue mimetics can also be generated by reaction with imidoesters, such as methyl picolinimidate, pyridoxal phosphate, pyridoxal, chloroborohydride, trinitrobenzenesulfonic acid, O-methylisourea, 2,4, pentanedione, and transamidase-catalyzed reactions with glyoxylate.

Methionine mimetics can be generated by reaction with methionine sulfoxide. Proline mimetics of include, for example, pipecolic acid, thiazolidine carboxylic acid, dehydroproline, 3- or 4-methylproline, and 3,3,-dimethylproline.

One or more residues can also be replaced by an amino acid (or peptidomimetic residue) of the opposite chirality. Thus, any amino acid naturally occurring in the L-configuration (which can also be referred to as R or S, depending upon the structure of the chemical entity) can be replaced with the same amino acid or a mimetic, but of the opposite chirality, referred to as the D-amino acid, but which can additionally be referred to as the R- or S-form.

As will be appreciated by one skilled in the art, the peptidomimetics of the present invention can also include one or more of the modifications described herein for derivatized peptides, e.g., a label, one or more post-translational modifications, or cell-penetrating sequence.

Also included with the scope of the invention are peptides and peptidomimetics that are substantially identical to a sequence set forth herein, in particular SEQ ID NO:1 or SEQ ID NO:19. The term “substantially identical,” when used in reference to a peptide or peptidomimetic, means that the sequence has at least 75% or more identity to a reference sequence (e.g., 80%, 85%, 90%, 95%, 96%, 97%, 98%, 99%). The length of comparison sequences will generally be at least 6 amino acids, but typically more, at least 8 to 10, 8 to 15, or 8 to 20 residues.

The peptides, derivatives and peptidomimetics can be produced and isolated using any method known in the art. Peptides can be synthesized, whole or in part, using chemical methods known in the art (see, e.g., Caruthers (1980) Nucleic Acids Res. Symp. Ser. 215-223; Horn (1980) Nucleic Acids Res. Symp. Ser. 225-232; and Banga (1995) Therapeutic Peptides and Proteins, Formulation, Processing and Delivery Systems, Technomic Publishing Co., Lancaster, Pa.). Peptide synthesis can be performed using various solid-phase techniques (see, e.g., Roberge (1995) Science 269:202; Merrifield (1997) Methods Enzymol. 289:3-13) and automated synthesis may be achieved, e.g., using the ABI 431A Peptide Synthesizer (Perkin Elmer) in accordance with the manufacturer's instructions.

Individual synthetic residues and peptides incorporating mimetics can be synthesized using a variety of procedures and methodologies known in the art (see, e.g., Organic Syntheses Collective Volumes, Gilman, et al. (Eds) John Wiley & Sons, Inc., NY). Peptides and peptide mimetics can also be synthesized using combinatorial methodologies. Techniques for generating peptide and peptidomimetic libraries are well-known, and include, for example, multipin, tea bag, and split-couple-mix techniques (see, for example, al-Obeidi (1998) Mol. Biotechnol. 9:205-223; Hruby (1997) Curr. Opin. Chem. Biol. 1:114-119; Ostergaard (1997) Mol. Divers. 3:17-27; and Ostresh (1996) Methods Enzymol. 267:220-234). Modified peptides can be further produced by chemical modification methods (see, for example, Belousov (1997) Nucleic Acids Res. 25:3440-3444; Frenkel (1995) Free Radic. Biol. Med. 19:373-380; and Blommers (1994) Biochemistry 33:7886-7896).

Alternatively, peptides of this invention can be prepared in recombinant protein systems using polynucleotide sequences encoding the peptides. By way of illustration, a nucleic acid molecule encoding a peptide of the invention is introduced into a host cell, such as bacteria, yeast or mammalian cell, under conditions suitable for expression of the peptide, and the peptide is purified or isolated using methods known in the art. See, e.g., Deutscher et al. (1990) Guide to Protein Purification: Methods in Enzymology Vol. 182, Academic Press.

It is contemplated that the peptides and mimetics disclosed herein can be used as lead compounds for the design and synthesis of compounds with improved efficacy, clearance, half-lives, and the like. One approach includes structure-activity relationship (SAR) analysis (e.g., NMR analysis) to facilitate the development of more efficacious agents. Agents identified in such SAR analysis or from agent libraries can then be screened for their ability to inhibit Aβ-induced synaptic depression mediated by PDZ-dependent recruitment of PTEN to dendritic spines.

For therapeutic applications, peptides and mimetics of the invention can be used as purified molecules (i.e., purified peptides, derivatives, or peptidomimetics), or in the case of peptides, be expressed from nucleic acids encoding said peptides. Such nucleic acids can, if desired, be naked or be in a carrier suitable for passing through a cell membrane (e.g., DNA-liposome complex), contained in a vector (e.g., plasmid, retroviral vector, lentiviral, adenoviral or adeno-associated viral vectors and the like), or linked to inert beads or other heterologous domains (e.g., antibodies, biotin, streptavidin, lectins, etc.), or other appropriate compositions. Thus, both viral and non-viral means of nucleic acid delivery can be achieved and are contemplated. Desirably, a vector used in accordance with the invention provides all the necessary control sequences to facilitate expression of the peptide. Such expression control sequences can include but are not limited to promoter sequences, enhancer sequences, etc. Such expression control sequences, vectors and the like are well-known and routinely employed by those skilled in the art.

For example, when using adenovirus expression vectors, the nucleic acid molecule encoding a peptide can be ligated to an adenovirus transcription/translation control complex, e.g., the late promoter and tripartite leader sequence. Alternatively, the vaccinia virus 7.5K promoter can be used. (see e.g., Mackett, et al. (1982) Proc. Natl. Acad. Sci. USA 79:7415-7419; Mackett, et al. (1984) J. Virol. 49:857-864; Panicali, et al. (1982) Proc. Natl. Acad. Sci. USA 79:4927-4931). Mammalian expression systems further include vectors specifically designed for “gene therapy” methods including adenoviral vectors (U.S. Pat. Nos. 5,700,470 and 5,731,172), adeno-associated vectors (U.S. Pat. No. 5,604,090), herpes simplex virus vectors (U.S. Pat. No. 5,501,979) and retroviral vectors (U.S. Pat. Nos. 5,624,820, 5,693,508 and 5,674,703 and WIPO publications WO 92/05266 and WO 92/14829).

Inhibitors of the invention (including nucleic acids encoding peptides) can be formulated with a pharmaceutically acceptable carrier at an appropriate dose. Such pharmaceutical compositions can be prepared by methods and contain carriers which are well-known in the art. A generally recognized compendium of such methods and ingredients is Remington: The Science and Practice of Pharmacy, Alfonso R. Gennaro, editor, 20th ed. Lippincott Williams & Wilkins: Philadelphia, Pa., 2000. A pharmaceutically acceptable carrier, composition or vehicle, such as a liquid or solid filler, diluent, excipient, or solvent encapsulating material, is involved in carrying or transporting the subject agent from one organ, or portion of the body, to another organ, or portion of the body. Each carrier must be acceptable in the sense of being compatible with the other ingredients of the formulation and not injurious to the patient.

Examples of materials which can serve as pharmaceutically acceptable carriers include sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

In addition to the active ingredient, a pharmaceutical composition of the invention may further include one or more additional pharmaceutically active agents or adjuvants conventionally used in the amelioration or treatment of β-amyloidogenic diseases. For example, the inhibitor here can be used in combination with a cholinesterase inhibitor (e.g., donepezil HCl, rivastigmine, galantamine or tacrine), memantine, vitamin E, an antidepressant (e.g., citalopram, fluoxetine, paroxeine, sertraline or trazodone), an anxiolytic (e.g., lorazepam or oxazepam), or an antipsychotic (e.g., aripiprazole, clozapine, haloperidol, olanzapine or risperidone).

Pharmaceutical compositions that are useful in the methods of the invention may be prepared, packaged, or sold in formulations suitable for oral, rectal, vaginal, parenteral, topical, pulmonary, intranasal, buccal, ophthalmic, intrathecal, or another route of administration. Other contemplated formulations include nanoparticles and liposomal preparations containing the active ingredient. Controlled- or sustained-release formulations of a pharmaceutical composition of the invention may also be made using conventional technologies.

As used herein, “parenteral administration” of a composition includes any route of administration characterized by physical breaching of a tissue of a subject and administration of the pharmaceutical composition through the breach in the tissue. Parenteral administration thus includes, but is not limited to, administration of a pharmaceutical composition by direct injection of the composition, by application of the composition through a surgical incision, by application of the composition through a tissue-penetrating non-surgical wound, and the like. In particular, parenteral administration is contemplated to include, but is not limited to, intraventricular (into the brain's ventricles), subcutaneous, intraperitoneal, intramuscular, intrasternal injection, and kidney dialytic infusion techniques.

A pharmaceutical composition of the invention may be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage. The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

The selected dosage level of an agent will depend upon a variety of factors including the activity of the particular agent employed, the route of administration, the time of administration, the rate of excretion or metabolism of the particular agent being employed, the duration of the treatment, other drugs, compounds and/or materials used in combination with the particular agent employed, the age, sex, weight, condition, general health and prior medical history of the patient being treated, and other factors well-known in the medical arts.

A physician having ordinary skill in the art can readily determine and prescribe the effective amount of the pharmaceutical composition required based upon the administration of similar compounds or experimental determination. For example, the physician could start doses of an agent at levels lower than that required in order to achieve the desired therapeutic effect and gradually increase the dosage until the desired effect is achieved. This is considered to be within the skill of the artisan and one can review the existing literature on a specific agent or similar agents to determine optimal dosing.

Based upon the findings that a peptide derived from the C-terminus of PTEN blocks Aβ-induced synaptic depression mediated by PDZ-dependent recruitment of PTEN and improves spatial learning in an animal model of Alzheimer's Disease, this invention is a method for mitigating or alleviating synaptic and cognitive deficits associated with a β-amyloidogenic disease using a peptide or mimetic described herein. As used herein, the terms “mitigating” or “alleviating” are meant to indicate delaying or even permanently delaying (i.e., preventing) development of synaptic and cognitive deficits and/or a reduction in the severity of synaptic and cognitive deficits that will, or are expected to, develop. The terms further include ameliorating existing symptoms or preventing additional symptoms. Therefore, the method of the invention encompasses applications to delay or arrest development of β-amyloidogenic disease in a subject at risk for such a disease. For instance, subjects with a genetic predisposition to Alzheimer's are suitable candidates for treatment according to the methods of the invention. The methods of the invention also encompass therapeutic treatment of a β-amyloidogenic disease in a subject diagnosed with such a disease. Advantageously, a peptide or mimetic inhibitor of the invention may reverse cognitive dysfunction and improve memory, such as spatial memory, and learning in a subject with Alzheimer's disease. Assays for determining the effectiveness of the peptide or mimetic of this invention include, but are not limited to, spatial learning tasks, memory tests and the like.

Diseases that may be treated by the method of the invention are β-amyloidogenic diseases. β-amyloidogenic diseases are characterized by the presence of Aβ plaques or deposits. For instance, Alzheimer's disease is characterized by mature senile plaques composed of Aβ in extracellular regions of the brain. β-Amyloidogenic diseases include, but are not limited to, Alzheimer's disease, Down's syndrome, mild cognitive impairment (MCI), cerebral amyloid angiopathy and hereditary cerebral hemorrhage with amyloidosis-Dutch type and -Icelandic type. In one embodiment of the invention, the β-amyloidogenic disease is Alzheimer's disease. Subjects suitable for treatment using the method of the invention are mammals, including humans.

The present invention is also directed to a kit to prepare and administer a composition containing a peptide or mimetic inhibitor that selectively blocks PDZ-dependent recruitment of PTEN into dendritic spines. The kit is useful for practicing the inventive method of treatment of β-amyloidogenic diseases such as Alzheimer's disease. The kit is an assemblage of materials or components, including at least one of the inventive compositions and a pharmaceutically acceptable carrier. Thus, in some embodiments, the kit contains a peptide derivative having the sequence N-myristoyl-QHSQITKV (SEQ ID NO:2) or N-myristoyl-QHTQITKV (SEQ ID NO:25), and a pharmaceutically acceptable carrier.

The exact nature of the components configured in the inventive kit depends on its intended purpose. For example, some embodiments are configured for the purpose of treating Alzheimer's disease. In one embodiment, the kit is configured particularly for the purpose of treating mammalian subjects. In another embodiment, the kit is configured particularly for the purpose of treating human subjects.

Instructions for use may be included in the kit. “Instructions for use” typically include a tangible expression describing the technique to be employed in using the components of the kit to effect a desired outcome, such as to monitor the improvement in cognitive function, memory and learning in a subject. Optionally, the kit also contains other useful components, such as, diluents, buffers, pharmaceutically acceptable carriers, syringes, catheters, applicators, pipetting or measuring tools, bandaging materials or other useful paraphernalia as will be readily recognized by those of skill in the art.

The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit. The packaging material is constructed by well-known methods, preferably to provide a sterile, contaminant-free environment. As used herein, the term “package” refers to a suitable solid matrix or material such as glass, plastic, paper, foil, and the like, capable of holding the individual kit components. The packaging material generally has an external label which indicates the contents and/or purpose of the kit and/or its components.

The invention is described in greater detail by the following non-limiting examples.

### Example 1: Materials and Methods

Animals.

Wild-type littermates were used as controls in each of the experiments involving transgenic mice. At weaning, the mice were genotyped from tail biopsies by means of polymerase chain reaction. The following mouse lines were used in this study:

APP/PS1 Mice.

Double transgenic (B6-Cg-Tg(APPswe, PSEN1dE9)85Dbo-J) mice were used for behavioral and biochemical experiments (male, age, 5 months at the end of the experiment). PCR-genotyping was carried out with three specific sense primers for PS1 (5′-CAGGTGGTGGAGCAAGATG; SEQ ID NO:26), APP (5′-CCGAGATCTCTGAAGTGAAGATGGATG; SEQ ID NO:27), and PrP (5′-CCTCTTTGTGACTATGTGGACTGATGTCGG; SEQ ID NO:28), and one common antisense primer matching the sequence within PrP (5′-GTGGATACCCCCTCCCCCAGCCTAGACC; SEQ ID NO:29) (Lesuisse, et al. (2001) Hum. Mol. Genet. 10:2525-2537). The PCR genotyping results were confirmed by histology using Thioflavin-S stain and by measurements of Aβ monomers (42 and 40) with ELISA.

PTEN^{tg} Mice.

Transgenesis procedures to obtain these mice have been described (Ortega-Molina, et al (2012) Cell Metabol. 15:382-394). Genetic background: C57BL6/CBA (75%:25%). Age, 5-6 months old.

The Pten^{tm(Q399stop)amc} (abbreviated as Pten^{ΔPDZ}) knockin mouse strain was generated by homologous recombination in iTL1 129S6/SvEvBrdTac(129Sv)-derived embryonic stem cells. The PDZ-binding domain was deleted by substituting codon 399 (CAA) with a stop codon (TAA). Chimeric founders were crossed once with C57BL/6J (The Jackson Laboratory, Bar Harbor, Me.) to generate heterozygous offspring, which were then mated with B6.FVB-Tg(EIIa-cre)C5379Lmgd/J mice (The Jackson Laboratory, Bar Harbor, Me.) to remove the Neomycin gene cassette. Progenies were backcrossed 10 generations to C57BL/6 background. Homozygous mice were generated by crossing heterozygous animals. Genotyping was carried by Polymerase Chain Reaction (PCR) using the following primers: 5′-GCTGAAGTGGCTGAAGAGCTCTGA-3′ (SEQ ID NO:30) and 5′-TTGAGTGAAACTGATGAGGTATGG-3′ (SEQ ID NO:31). Wild-type allele yielded a 1545 base pair (bp) fragment while the knockout allele generated a 1724 bp product due to the presence of a 179 bp sequence from the knockin vector following cre-mediated recombination.

PTEN Inhibition In Vivo with Osmotic Pumps.

APP/PS1 and wild-type mice were anesthetized with isofluorane, and i.c.v. delivery cannulas (brain alzet kit III) were implanted with a stereotaxic frame (KOPF Instruments) at the following coordinates according to the bregma: AP, −0.5 mm; ML, 1 mm; and DV, −2.2 mm. Osmotic minipumps (Alzet) were filled with artificial cerebrospinal fluid (aCSF) with or without PTEN inhibitor VO-OHpic (2.5 μM) and equilibrated in 0.9% NaCl at 37° C. for 48 hours. They were attached to the i.c.v. cannula tubing and subcutaneously implanted at the back. After 21 days, behavioral testing was started as described herein. One month after implantation, mice were sacrificed and the brains were sliced stained with Nissl to verify the location of the cannula. Animal manipulation and data analysis was carried out blind with respect to genotype and treatment.

Antibodies.

6E10 antibody (Covance) was used for immunoprecipitation and detection of secreted APP as well as detection of synthetic Aβ with western blot. APP full-length and APP C-terminal fragments were precipitated with APP C-terminal antibody (Sigma), monoclonal mouse anti-APP (MAB348, Millipore). Secreted wild-type APP was detected with the specific antibody (39138, Covance) while the Swedish variant of APP was visualized using 6A1 antibody (IBL). NU-1, monoclonal mouse antibody was used for immunofluorescence. Other antibodies used for the western blots: anti-GFP (Roche) anti-Akt, phospho-Akt (T308), GSK3β and phospho-GSK3β (S9) (Cell Signaling), anti-PTEN (138G6, 9559, Cell Signaling), anti-tubulina (T6199 Cell Signaling).

Protofibrillar Aβ42 Preparation.

Lyophilized Aβ42 peptide was purchased from Keck Facility at Yale University (New Haven, Conn.). To obtain Aβ42 enriched in protofibrillar aggregates (Jan, et al. (2010) Nat. Protoc. 5:1186-1209), 1 mg of lyophilized Aβ was dissolve in 50 μl of 100% DMSO. This step was followed by addition of 800 μl of H₂O and of 10 μl of 2 M Tris-base solution (pH 7.6). The solution was then incubated at room temperature for 5 minutes and used immediately afterward.

Oligomeric Aβ42.

Lyophilized Aβ42 peptide was purchased from Invitrogen. The peptide was dissolved in water at 6 mg/ml, then diluted to 1 mg/ml with PBS and incubated at 37° C. for 36 hours and frozen in 5 μl aliquots.

Thioflavin-T Assay.

Freshly prepared Aβ42 was prepared as described for protofibrillar Aβ42. The Thioflavin-T (ThT) assay was carried out at 37° C., within a dark chamber in a 96-well plate. Each well contained 100 ng/μL of Aβ42, 20 μM ThT and 50 nM VO-OHpic or 15 nM bPV(HOpic). Fluorescence (λₑₓ=450 nm; λₑₘ=485 nm) was followed over time in a FLUOSTAR OPTIMA (BMG LabTech) fluorescence spectrophotometer. Samples were run in triplicates.

Electron Microscopy.

Aβ42 peptides (Invitrogen) were adsorbed onto ionized Collodion/carbon-coated copper grids and negatively stained with 2% aqueous uranyl acetate for 45 seconds. Grids were visualized on a JEM1010 transmission electron microscope (Jeol, Japan) and pictures were taken with a TEMCAM-F416 TVIPS digital camera (Gauting, Germany).

ELISA.

Hippocampal slices overexpressing Aβ were maintained for 3 days after virus injection with or without the PTEN inhibitor VO-OHpic, before culture medium was collected for measurements. Aβ40 and Aβ42 level were determined in solubilized hippocampal fractions, cultured hippocampal slices or conditioned media by ELISA according to the manufacturer's instructions (WAKO/Invitrogen) that specifically detect the C-terminus of Aβ40 and Aβ42, respectively.

Full Length APP and APP Processing Measurements.

Hippocampi from APP/PS1 mice or their wild-type littermates and organotypic hippocampal slices infected with APP_{swe/lnd}-EGFP/APP_{wt}-EGFP/APP_{MV}-EGFP were solubilized, and extracts were prepared for western blot analysis. Mouse brains or brain sections were homogenized on ice in RIPA buffer (1% TRITON X-100, 1% sodium deoxycholate, 0.1% SDS, 150 mM NaCl, 50 mM Tris-HCl, pH 7.2), using an ULTRATURRAX T25 (Janke & Kunkel). Samples were centrifuged at 15,000×g, and supernatants were used for protein determination. APPsα was detected in conditioned media of the respective slices using antibody 6E10 that selectively detects human APPsα. Levels of APP, CTFα and CTFβ were detected in lysates of the infected slices using APPCt antibody.

Electrophysiology.

Mice were anesthetized with sodium pentothal (20 mg/kg ip) and decapitated. The brain was rapidly removed to ice-cold, oxygenated, dissection solution. Coronal slices (300 μm) were made with a vibratome (LEICA VT1200S) and slices were moved to a recovery chamber containing aCSF at room temperature for at least 1.5 hour before recording.

A concentric bipolar platinum-iridium stimulation electrode and a low-resistance glass recording microelectrode filled with aCSF (3-4 MΩ resistance) were placed in CA1 stratum radiatum to record the extracellular field excitatory postsynaptic potentials (fEPSPs). For each slice, an input-output curve was recorded to compare basal synaptic transmission at different conditions. This curve was also used to set the baseline fEPSP at ≈30% (for LTP experiments) or ≈50% (for LTD experiments) of maximal slope. Baseline stimulation was delivered every 15 seconds (0.01 ms duration pulses) for at least 20 minutes before beginning the experiment to ensure stability of the response. LTP was induced by using θ-burst stimulation (4 pulses at 100 Hz, with the bursts repeated at 5 Hz and each tetanus including three 10-burst trains separated by 15 seconds). LTD was induced using 900 pulses at 1 Hz. Responses were recorded for 1 hour after induction of LTP or LTD.

Whole-Cell Electrophysiology.

The effect of recombinant protein expression on synaptic transmission was evaluated by simultaneous double whole-cell recordings from pairs of nearby infected and uninfected CA1 neurons, under voltage clamp, while stimulating presynaptic Schaffer collateral fibers (under this configuration, recombinant proteins are expressed exclusively in the postsynaptic neuron).

Construction of Recombinant Proteins and Expression.

The different versions of APP (APP_{wt}, APP_{swe/lnd} APP_{MV}) and EGFP were co-expressed using an internal ribosomal entry site (IRES) construct. The EGFP-tagged versions of PTEN have been described (Jurado, et al. (2010) EMBO J. 29:2827-2840). All constructs were prepared in pSinRep5 for expression using Sindbis virus. Recombinant proteins were expressed in hippocampal CA1 pyramidal neurons from organotypic slice cultures. Organotypic hippocampal slices were prepared from postnatal day 5-7 rats and cultured during 6-8 days.

Statistical Analysis.

Statistical differences were calculated according to non-parametric tests unless indicated otherwise. Comparisons between multiple groups were carried out with the Kruskal-Wallis ANOVA. When significant differences were observed, p values for pairwise comparisons were calculated according to two-tailed Mann-Whitney tests (for unpaired data) or Wilcoxon tests (for paired data).

Peptide Synthesis.

All peptides were prepared manually using Fmoc-based solid-phase synthesis protocols with HCTU as the coupling agent (Hood, et al. (2008) J. Pept. Sci. 14:97-101). Fluorescently-labeled peptides were prepared by adding a Fmoc-Lys residue (with Mtt side chain protecting group) to the N-terminus of the peptide while on resin, selectively removing the Mtt protecting group, and covalently attaching fluorescein through reaction with 5-FITC (fluorescein-5-isothiocyanate). All peptides were purified using semi-preparative, reverse-phase HPLC (RP-HPLC) using a C18 column with water-methanol mobile phase gradient, followed by lyophilization to yield white solids. Molecular mass of each purified peptide was confirmed by LCMS analysis (Shimadzu LCMS-2020).

Behavioral Experiments:

Novel-Object Location. This assay was chosen because it is not intrinsically stressful. This factor, in mice, crucially affects cognitive performance in other spatial learning paradigms (e.g., Morris water maze). The memory tests were composed of three phases—“habituation”, “sample” and “choice” trials. Mice were first habituated individually to an empty open-field box (35×35×15 high cm) for 30 minutes. A sample trial (object exposure) consisted of placing a mouse into the test box which contained two identical objects. The mouse was removed from the test box and after a delay (retention period) of 30 minutes the mouse was placed back into the test box for a choice trial. A choice trial consisted of switching the location of one of the objects (Novel-Object Location trial). A recognition index was calculated by dividing the total time spent exploring the displaced object by the total time spent exploring both objects during the test session. A recognition index of 0.5 would, therefore, correspond to equal exploration of both objects. Subjects were excluded from the analysis if they failed to explore both stimulus objects for a total of at least 10 seconds during either training or test sessions. One mouse was excluded from this study based on this criterion.

Behavioral Experiments:

Fear conditioning. In this test, mice form an association between a certain context (an experimental cage/tone) and an aversive event (a foot shock) that takes place in that context. When placed back into the context, mice exhibit a range of conditioned fear responses, including immobility (freezing). Training and testing took place in a rodent observation cage (30×37×25 cm) that was placed in a sound-attenuating chamber. In the training (conditioning), the mouse was exposed to the conditioning context (180 seconds) followed by a tone (CS, sec, 2 kHz, 85 dB). After termination of the tone, a footshock (US, 0.75 mA, 2 seconds) was delivered through a stainless steel grid floor. Mice received three footshocks with an intertrial interval of 60 seconds. The mouse was removed from the fear conditioning box 30 seconds after shock termination and returned to their home cages. Testing: In the contextual fear conditioning version, mice were placed back into the original training context for 8 minutes, during which no footshock was delivered. In the auditory-cued fear-conditioning version, animals were placed into a novel context (same cages, but with different walls, floor, and background odor), and, after a 3 minutes baseline period, they were continuously re-exposed to the tone (same characteristics as at conditioning) for 5 minutes, but in the two absence of shocks. The animals' behavior was scored by an observer blind to the treatment condition. Using a time-sampling procedure every 2 seconds, each mouse was scored blindly as either freezing or active at the instant the sample was taken. Freezing was defined as behavioral immobility except for movement needed for respiration.

Electrophysiology Field Recording.

Dissection solution (employed for slicing) composition: 10 mM D-glucose, 4 mM KCl, 26 mM NaHCO₃, 233.7 mM sucrose, 5 mM MgCl₂, 1:1000 Phenol Red. Artificial CSF (employed for recovery and recording) composition: 119 mM NaCl, 2.5 mM KCl, 1 mM NaH₂PO₄, 11 mM glucose, 1.2 mM MgCl₂, 2.5 mM CaCl₂. Osmolarity was adjusted to 290 Osm.

Voltage Clamp.

The external solution (aCSF) contained 119 mM NaCl, 2.5 mM KCl, 1 mM NaH₂PO₄, 11 mM glucose, 26 mM NaHCO₃, 4 mM MgCl₂, 4 mM CaCl₂, 100 μM picrotoxin and 2 μM 2-chloroadenosine, pH 7.4, and was gassed with 95% O₂ and 5% CO₂. Patch recording pipettes (4-7 MO) were filled with internal solution containing 115 mM CsMeSO₃, 20 mM CsCl, 10 mM HEPES, 2.5 mM MgCl₂, 4 mM Na₂-ATP, 0.4 mM Na-GTP, 10 mM sodium phosphocreatine and 0.6 mM EGTA, pH 7.25. Bipolar stimulating electrodes were placed over Schaffer collateral fibers between 250 and 300 μm from the CA1 recorded cells, and synaptic responses were evoked with single voltage pulses (200 μs, up to 30 V). Responses were collected at −60 mV and +40 mV and averaged over 50-100 trials. LTP was induced using a pairing protocol by stimulating Schaffer collateral fibers at 3 Hz for 1.5 minutes while depolarizing the postsynaptic cell at 0 mV. All electrophysiological data were collected with pCLAMP software (Molecular Devices). Immunohistochemistry For the immunostaining protocol, hippocampal slice cultures were fixed with 4% paraformaldehyde for 24 hours washed in PBS and blocked with 5% horse serum for 2 hours. Sections were then incubated for 48 hours at 4° C. with primary antibody. After 3 washes with PBS, slices were incubated for 2 hours at room temperature with the secondary antibody labeled with ALEXAFLUOR-594 (diluted 1:500 in 5% horse serum), washed, mounted, and imaged with a ZEISS LSM510 confocal microscope.

Preparation of Hippocampal Cultures and Expression of Recombinant Proteins.

For organotypic cultures, hippocampal slices were prepared from rat pups at postnatal day 5-6 and were cultured for 5 to 7 days. Primary hippocampal cultures were prepared from E18 rat embryo neurons (Kaech & Banker (2006) Nature Protocols 1:2406-2415). For live imaging experiments, 6×10⁴ cells were plated into 3-cm plastic dishes with a 15 mm coverslip, and coated with poly-L-lysine (1 mg/ml). For biochemical analysis, 7.5×10⁵ cells were plated into 10-cm plastic dishes and coated with poly-L-lysine (0.1 mg/ml). Neurons were kept under 5% CO₂ at 37° C. in neurobasal medium plus B27 supplement and GLUTAMAX (Gibco) until DIV5. Then medium was replaced with Neurobasal medium plus B27 without GLUTAMAX. One to two days before use, hippocampal neurons were exposed to a solution containing a Sindbis virus carrying the genes of interest.

Microscopy and Image Acquisition.

Confocal imaging was performed with a laser scanning confocal microscope (LSM510 META, ZEISS) using a 40× or 63× oil-immersion objective (numeric aperture, 1.3 or 1.4, respectively) and either 488 nm or 561 nm excitation. Serial optical sections were acquired at 0.4-1 μm intervals through given neurons or dendritic arbor. For time-lapse experiments, laser intensity, gain, offset, and contrast settings were chosen to optimize visualization of spines (while avoiding saturation of the signal) before data acquisition, and were not later altered within individual experiments. Imaging solution for time-lapse imaging was composed of: 120 mM NaCl, 3 mM KCl, 2 mM CaCl₂, 2 mM MgCl₂, 10 mM glucose and 10 mM HEPES. Protofibrillar Aβ (4 μM) was added after a baseline acquisition of at least 8 minutes. Images were analyzed with Image J. For each individual spine, signal intensity for spine and adjacent dendrite was computed to generate the spine to dendrite EGFP ratio. Spine/dendrite ratio was determined for each spine every 2 minutes and then was normalized to the baseline of the same spine before the addition of Aβ. Only spines with a stable baseline (change in the spine/dendrite ratio less than 5% during baseline) were included in the analysis.

Western Blot.

The homogenization buffer was composed of the following (unless indicated otherwise): 10 mM HEPES, 150 mM NaCl, 10 mM EDTA, 1% TRITON, pH 7.4. Protease 4 Inhibitor Cocktail Tablets “Complete mini” (1:7) and Phosphatase Inhibitor Cocktail Tablets “PHOSSTOP” (1:10) were prepared by the manufacture's (Roche) instructions.

### Example 2: PTEN Inhibition Rescues Cognitive Function in APP/PS1 Mice

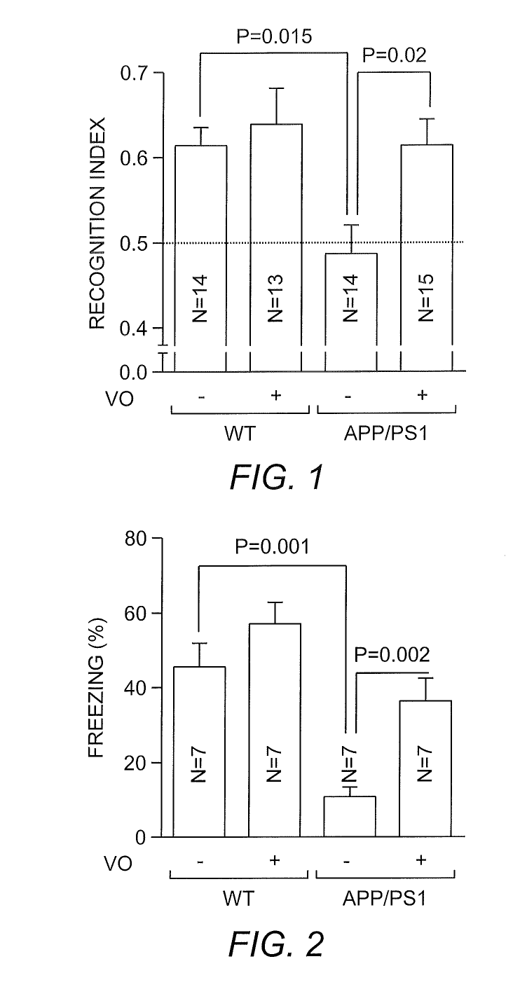

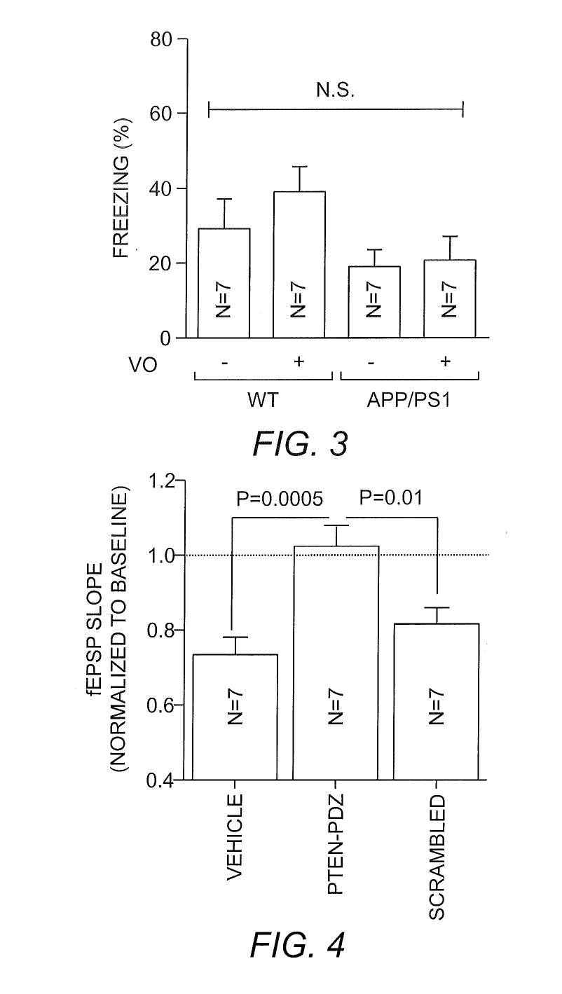

It was posited that if early Aβ-induced cognitive impairment results from a general synaptic bias toward LTD events (Selkoe (2002) Science 298:789-791), dampening this form of synaptic plasticity might prevent cognitive decay. Therefore, it was determined whether in vivo inhibition of PTEN, a lipid phosphatase that is involved in LTD (Jurado, et al. (2010) EMBO J. 29:2827-2840), would rescue cognitive deficits in APP/PS1 mice. This mouse model displays an accelerated AD-type phenotype due to mutant Amyloid Precursor Protein (APP) and presenilin 1 (PS1) transgenes (Holcomb, et al. (1998) Nat. Med. 4:97-100). The specific PTEN inhibitor VO-OHpic (Rosivatz, et al. (2006) ACS Chem. Biol. 1:780-790) or vehicle (artificial cerebrospinal fluid, ACSF) were infused during 3-4 weeks by osmotic minipumps into the brain ventricles of 4-month-old transgenic mice and their wild-type littermates. Mice were subject to two hippocampal-dependent cognitive tasks: novel object-location and contextual fear conditioning (these experiments were carried out blind with respect to genotype and treatment). As shown in FIG. 1, vehicle-treated APP/PS1 mice showed a significant impairment in the recognition of the novel object location (P=0.015, compared to vehicle-infused wild-type mice). Notably, while the infusion of VO-OHpic did not affect the performance of wild-type mice, treated APP/PS1 mice showed a significant improvement in this spatial learning task (P=0.02, compared to vehicle-infused APP/PS1 mice), to the extent that their recognition index was similar to that of wild-type mice. It should be noted that this result was replicated in 2 batches of mice coming from different colonies and raised in different animal houses. Similarly, APP/PS1 mice were impaired in contextual fear conditioning (P=0.001) (FIG. 2), and VO-OHpic significantly improved their performance (P=0.002), without altering that of wild-type mice. Importantly, a hippocampal-independent version of this task (auditory-cued fear conditioning) did not show significant difference between wild-type and APP/PS1 mice, regardless of VO-OHpic treatment (FIG. 3). Cannula placement was confirmed with Nissl staining at the end of the experiment. Only those mice with a cannula placement in the lateral ventricle were used for analysis (mice were PCR-genotyped twice before implantation of the pumps.

After the end of the last test, the levels of APP and its C-terminal fragments (western blot), Aβ monomers (ELISA), and SDS-resistant. Aβ assemblies (SDS-PAE) were measured from the hippocampus of each mouse. No changes were detected in any of these parameters following VO-OHpic treatment. Thus, in vivo PTEN inhibition rescues cognitive function in APP/PS1 without detectable changes in Aβ levels or APP processing.

### Example 3: PTEN Activity Mediates Aβ42-Induced Synaptic Depression and LTP Impairment

In order to evaluate the synaptic basis of the cognitive protection achieved by PTEN inhibition, it was determined whether PTEN is required for Aβ-induced synaptic depression. To this end, two sources of Aβ were used: water-soluble, oligomeric assemblies of synthetic Aβ, and Aβ secreted from neurons expressing a mutant form of APP (human APP with the Swedish/London double mutation).

For the first approach, protofibrillar Aβ was prepared by promoting aggregation under high-salt conditions (258 μM Aβ42, room temperature incubation (Jan, et al. (2010) Nat. Protoc. 5:1186-1209). The kinetics of Aβ42 aggregation were followed by thioflavin T fluorescence and SDS-PAGE western blot analysis with the N-terminal anti-Aβ antibody 6E10. Also, the heterogeneous nature of these Aβ42 assemblies was evidenced by electron microscopy. Synaptic function was assessed by electrophysiological recordings of field excitatory postsynaptic potential (fEPSPs) between hippocampal CA3 and CA1 cells from acute slices prepared from 5-month-old mice. The results indicated a ≈60% decrease in the magnitude of synaptic transmission (input-output curve) when slices were incubated for 2 hours in aCSF containing pathogenic protofibrillar Aβ42 species (1 μM). This decrease in the basal synaptic transmission was prevented when Aβ was added to slices that were preincubated with the PTEN inhibitor (50 nM VO-OHpic) for 1 hour (VO-OHpic and Aβ were also present in the perfusion solution during electrophysiological recordings). VO-OHpic alone had no effect on basal synaptic transmission. The effectiveness of VO-OHpic as a PTEN inhibitor and its specificity versus Tyr phosphatases were evidenced from the upregulation of the PIP₃ downstream effector phospho-Akt and the lack of effect on phospho-Tyr levels in acute hippocampal slices. The potency and specificity of this inhibitor have been documented previously (Rosivatz, et al. (2006) ACS Chem. Biol. 1:780-790).

In addition to synaptic depression, Aβ oligomers have also been shown to impair LTP expression (Shankar, et al. (2008) Nat. Med. 14:837-842; Selkoe (2008) Behav. Brain Res. 192:106-113; Welsby, et al. (2007) Neuropharmacology 53:188-195; Klyubin, et al. (2004) Euro. J. Neurosci. 19:2839-2846; Wang, et al. (2004) J. Neurosci. 24:3370-3378; Chen, et al. (2002) Neurobiol. Learn. Mem. 77:354-371). Indeed, it was found that protofibrillar Aβ42 strongly inhibited LTP expression in the CA1 hippocampal region (slices were incubated in 1 μM Aβ42 for 2 hours prior to recording and during the recording period). Similar to the results with basal transmission, preincubation with the PTEN inhibitor (50 nM VO-OHpic, 1 hour before addition of Aβ42) rescued the expression of LTP. Thus, VO-OHpic prevented Aβ42-induced impairments in both basal synaptic transmission and LTP, suggesting that PTEN activity mediates these synaptic pathologies. Importantly, using the thioflavin-T (ThT) fluorescence assay, it was demonstrated that VO-OHpic (as well as bPV(HOpic), a chemically related PTEN inhibitor) does not alter Aβ42 aggregation. Thus, the observed rescue of basal synaptic transmission, LTP and cognition, with VO-OHpic was not likely due to an altered aggregation state of Aβ upon PTEN inhibition.

Since both PTEN (Jurado, et al. (2010) EMBO J. 29:2827-2840) and protofibrillar Aβ42 produce synaptic depression, it was determined whether they act via common mechanisms, using an occlusion strategy. This analysis was carried out using transgenic mice designed to possess an increased gene dosage of PTEN, while preserving its natural pattern of gene expression (Ortega-Molina, et al (2012) Cell Metabol. 15:382-394). Indeed, the slope of the electrically evoked field potential was significantly reduced in acute hippocampal slices taken from PTEN^{tg} mice, when compared to slices from wild-type littermates. Notably, no further depression was observed upon Aβ incubation. This occlusion effect implies that PTEN and Aβ act in a common pathway eventually leading to synaptic depression.

### Example 4: PTEN Inhibition Rescues AMPAR-Mediated Transmission in Neurons Overproducing Aβ

As a complementary approach to the use of synthetic Aβ, the role of PTEN in Aβ-induced synaptic depression was further evaluated by expressing an APP gene carrying the Swedish and London mutations (APP_{swe/lnd}) in rat hippocampal slice neurons using a viral expression system (Gerges, et al. (2005) Meth. Enzymol. 403:153-166). APP_{swe/lnd} was co-expressed with enhanced green fluorescent protein to allow the identification of the infected neuron during electrophysiological recordings. Expression of APP_{swe/lnd} in CA1 neurons resulted in substantially higher expression of APP, APP C-terminal fragments and accumulation of Aβ (as monitored with immunohistochemistry, ELISA, and western blot). To test the effects of APP_{swe/lnd}-expression on synaptic transmission, the synaptic responses evoked onto adjacent pairs of simultaneously recorded CA1 pyramidal neurons were compared, where only one cell expresses the recombinant protein. After 20-48 hour expression, APP_{swe/lnd}-expressing neurons showed 40% depression of AMPA receptor synaptic responses relative to uninfected cells. These results indicated that APP_{swe/lnd}-expression in CA1 neurons depresses glutamatergic synaptic transmission (at these expression times, depression was specific for AMPAR versus NMDA responses). To note, uninfected cells adjacent to APP_{swe/lnd}-expressing cells were also expected to be affected by the secreted Aβ (Kamenetz, et al (2003) Neuron 37:925-937). Therefore, the depression observed in the APP_{swe/lnd}-expressing neuron only implied that the Aβ-producing cell was affected to a larger extent than its uninfected neighbor. Importantly, this depression was due to β-secretase (BACE) processing of APP, and not just to protein overexpression or virus infection, because overexpression of a mutant APP with little susceptibility to BACE cleavage (APP M596V; Citron, et al (1995) Neuron 14:661-670) did not produce synaptic depression.

To test the role of PTEN in this form of depression, APP_{swe/lnd} was expressed in the presence of either 15 nM bPV(HOpic) or 50 nM VO-OHpic, which are two chemically related PTEN inhibitors (Rosivatz, et al. (2006) ACS Chem. Biol. 1:780-790). Under these conditions, AMPAR-mediated excitatory postsynaptic currents (EPSCs) were no longer depressed in APP_{swe/lnd}-expressing neurons. In fact, AMPAR responses were significantly increased as compared to uninfected cells. This set of experiments, using Aβ produced by neurons, confirmed that inhibition of PTEN phosphatase activity rescued synaptic depression. Therefore, these results indicate that Aβ-induced synaptic depression requires PTEN activity. Importantly, PTEN inhibition did not alter recombinant APP expression, formation of C-terminal APP fragments or Aβ production.

### Example 5: PTEN Blockade Protects from Neuron-Secreted Aβ

The results herein indicated that PTEN inhibition protects from synaptic depression induced by APP_{swe/lnd} expression. However, this form of depression may in principle be due to Aβ exposure, to intracellular fragments derived from APP processing, or a combination thereof. To test the role of PTEN specifically in the effects of neuron-secreted Aβ, synaptic dysfunctions were monitored in untransfected cells neighboring APP_{swe/lnd}-expressing neurons. Aβ produced by APP_{swe/lnd}-EGFP infected neurons was detected on the cell surface of infected neurons and was secreted into the slice culture medium, together with APP N-terminal fragments (APPsα), in an amount independent of VO-OHpic treatment, as determined with ELISA and western Blot. LTP was induced in CA1 individual neurons by pairing presynaptic stimulation (3 Hz, 300 pulses) with postsynaptic depolarization (0 mV). LTP was efficiently induced in neurons in control slices not injected with APP_{swe/lnd}-EGFP virus. In contrast, APP_{swe/lnd}-EGFP infected cells did not show any potentiation. Notably, uninfected neurons adjacent to APP_{swe/lnd}-EGFP infected cells (<30 μm) did not show any LTP either, likely due to their exposure to extracellular Aβ secreted from neighboring infected cells (Kamenetz, et al (2003) Neuron 37:925-937). Thus, Aβ prevented the induction of LTP both in APP_{swe/lnd}-expressing neurons and in adjacent uninfected neurons. Injected and uninjected slices were incubated with the PTEN inhibitor VO-OHpic for 26-48 hours, during APP_{swe/lnd}-EGFP expression. It was observed that while VO-OHpic did not affect the magnitude of LTP in control slices (not exposed to APP_{swe/lnd}-EGFP virus), APP_{swe/lnd}-EGFP-infected and uninfected neurons exposed to Aβ recovered the ability to express significant LTP (uninfected cells: potentiation of 339%, P=0.0006; APP_{swe/lnd}-EGFP infected cells: potentiation of 220%, P=0.01). Thus, PTEN inhibition rescued LTP in neurons exposed to extracellular Aβ secreted from APP_{swe/lnd}-expressing neurons.

### Example 6: Cell-Autonomous Blockade of PTEN Activity Protects Against Aβ Toxicity

To test the role of PTEN as a cell-autonomous factor in Aβ synaptic malfunction, a catalytically dead form of PTEN was expressed in postsynaptic CA1 neurons. This catalytically dead form of PTEN acts as a dominant negative mutant (PTEN-C124S; Maehama & Dixon (1998) J. Biol. Chem. 273:13375-13378) and blocks LTD (Jurado, et al. (2010) EMBO J. 29:2827-2840). Neurons expressing this catalytically dead form of PTEN were exposed to extracellular Aβ by expression of APP_{swe/lnd}-EGFP in neighboring neurons or incubation with 1 μM oligomeric synthetic Aβ42. To evaluate the effect of extracellular Aβ on individual postsynaptic neurons in which PTEN activity is blocked, evoked AMPAR and NMDAR responses were compared between PTEN-C124S-expressing neurons and non-infected neighboring control neurons at −60 mV and +40 mV. Importantly, under these settings both PTEN-C124S and control neurons are exposed to extracellular Aβ (either synthetic or secreted from neighboring APP_{swe/lnd}-expressing neurons). The results of this analysis indicated that the AMPAR- and NMDAR-mediated synaptic responses were significantly increased in PTEN-C124S-expressing neurons, as compared to uninfected cells. This indicates that suppression of PTEN activity in individual postsynaptic neurons protects them from Aβ-induced synaptic depression (resulting in apparent potentiation with respect to uninfected neurons exposed to Aβ). Therefore, these experiments confirm that PTEN activity is required for the synaptic depression triggered by Aβ.

It was subsequently determined whether PTEN-C124S expression would also restore LTP capacity in individual neurons exposed to 1 μM synthetic Aβ42. It was found that PTEN-C124S-infected neurons exposed to Aβ42 expressed significant LTP, in contrast to uninfected cells exposed to Aβ from neighboring Aβ-producing neurons. Thus, suppression of PTEN phosphatase activity in a cell-autonomous manner in postsynaptic neurons protects from the synaptic plasticity impairment produced by either synthetic or neuron-secreted Aβ.

### Example 7: PTEN is Recruited to Spines in a PDZ-Dependent Manner Upon Exposure to Aβ

Based upon the results herein, it was posited that PTEN, besides being a mediator of Aβ-induced synaptic dysfunction, may also be engaged as a regulated component in the process. It has been shown that NMDAR-dependent LTD results in PTEN anchoring to the postsynaptic terminal (Jurado, et al. (2010) EMBO J. 29:2827-2840). To determine whether Aβ induces a similar PTEN redistribution, EGFP-tagged PTEN (EGFP is fused to the N-terminus of PTEN) was expressed in primary hippocampal neurons for 15-18 hours. Time-lapse imaging of infected CA1 neurons before Aβ application showed widespread and homogenous distribution of EGFP-PTEN, including distal dendrites and spines. Importantly, following the addition of synthetic protofibrillar Aβ42 (4 μM) EGFP-PTEN rapidly accumulated in spine heads and remained there for at least 1 hour.

The involvement of PTEN in NMDAR-dependent LTD and its anchoring to the postsynaptic terminal is dependent upon the C-terminal PDZ binding motif of PTEN (-ITKV*; SEQ ID NO:32) (Jurado, et al. (2010) EMBO J. 29:2827-2840). To determine whether Aβ42-induced mobilization of PTEN into spines also requires PDZ-dependent interactions, time-lapse imaging was carried out with neurons expressing a truncated form of PTEN lacking its PDZ-binding motif (EGFP-PTEN-ΔPDZ) (Jurado, et al. (2010) EMBO J. 29:2827-2840). It was observed that, compared with the full-length PTEN, EGFP-PTEN-ΔPDZ was mobilized into spines more slowly and to a lesser extent. Thus, Aβ42 triggers the insertion of PTEN into spines through a PDZ-dependent interaction.

Since PTEN appears to be a regulated target in Aβ toxicity, it was determined whether Aβ exposure (by APP_{swe/lnd} overexpression) could globally alter the PIP₃ pathway. This was evaluated by monitoring downstream effectors: the phosphorylated forms of Akt and GSK3β. Unchanged levels of phospho-Akt (T308), phospho-GSK3β (S9) and total levels of these kinases in APP_{swe/lnd}-EGFP infected slices was observed 72 hours post-infection. Similarly, no significant alterations were detected in the levels of these downstream effectors in APP/PS1 mice or in human Alzheimer's disease hippocampal samples in which high levels of Aβ are chronically present. These combined findings indicated that Aβ overexpression does not globally alter the PIP₃ pathway, but relies on local redistributions of PIP₃ regulators, such as PTEN translocation to synaptic sites.

### Example 8: PTEN-PDZ Interaction is Essential to Aβ-Induced Synaptic Toxicity

To establish the role of PTEN-PDZ interaction in the disease mechanisms, a new knock-in mouse model was developed in which PTEN lacks the last 5 amino acids (-QITKV; SEQ ID NO:33), therefore removing the C-terminal PDZ binding motif (PTEN^{ΔPDZ} mice). Total levels of PTEN protein and global activation of the PIP₃ pathway (as reported from phospho-Akt levels) were normal in these animals. Acute hippocampal slices from these mice and from their wild-type littermates (5-months-old) were treated with 1 μM synthetic protofibrillar Aβ42 for 2 hours and fEPSPs were recorded to yield input-output curves. Basal synaptic transmission was similar in wild-type and PTEN^{ΔPDZ} animals. However, while incubation with Aβ42 significantly depressed the input-output curve in wild-type slices, slices taken from PTEN^{ΔPDZ} mice were completely resistant to Aβ treatment, and basal synaptic transmission remained similar to untreated slices. These results indicate that Aβ-triggered synaptic depression relies on PTEN interactions with PDZ proteins.

It was also determined whether this was the case using neuron-produced Aβ, via APP_{swe/lnd} expression. Using organotypic slice cultures from PTEN^{ΔPDZ} mice and from their wild-type littermates, synaptic responses evoked onto adjacent pairs of simultaneously recorded neurons were compared where only one neuron expresses APP_{swe/lnd}-EGFP. As expected from similar experiments performed in rat organotypic slice cultures, APP_{swe/lnd}-expressing neurons showed significant depression of AMPA receptor synaptic responses relative to uninfected cells in wild-type slices. In contrast, APP_{swe/lnd}-expression in neurons from PTEN^{ΔPDZ} mice did not depress AMPAR-mediated synaptic transmission, similar to the results with acute slices and synthetic Aβ.

Besides depression of basal synaptic transmission and impairment of LTP, Aβ has been shown to enhance LTD (Li, et al. (2009) Neuron 62:788-801). Therefore, it was tested whether PTEN-PDZ interaction also mediates this altered form of synaptic plasticity. To this end, LTD was induced in acute hippocampal slices with an NMDAR-dependent protocol (900 pulses at 1 Hz), under partial NMDAR blockade (30 μM AP5) (Li, et al. (2009) Neuron 62:788-801). The results of this analysis indicated that Aβ42 facilitated LTD in wild-type slices. In contrast, slices from PTEN^{ΔPDZ} mice failed to show LTD, and there was no LTD facilitation upon Aβ treatment. Thus, enhancement of NMDAR-dependent LTD by Aβ42 requires PTEN-PDZ interactions.

Together, these experiments indicate that removal of PTEN-PDZ interactions turns neurons resistant to Aβ-induced synaptic alterations.

### Example 9: An N-Myristoylated Octapeptide Containing a PTEN-PDZ Binding Motif Protects Against Aβ-Induced Synaptic Depression

To target PDZ-dependent PTEN interactions, a peptide corresponding to the last eight amino acids of rat/mouse PTEN was generated with the addition of myristic acid to augment cell permeability (“PTEN-PDZ,” N-myristoyl-QHSQITKV (SEQ ID NO:2)). A control peptide, with the same amino acid composition, but in a scrambled sequence, was also synthesized (“scrambled,” N-myristoyl-SVHTIQKQ (SEQ ID NO:34).

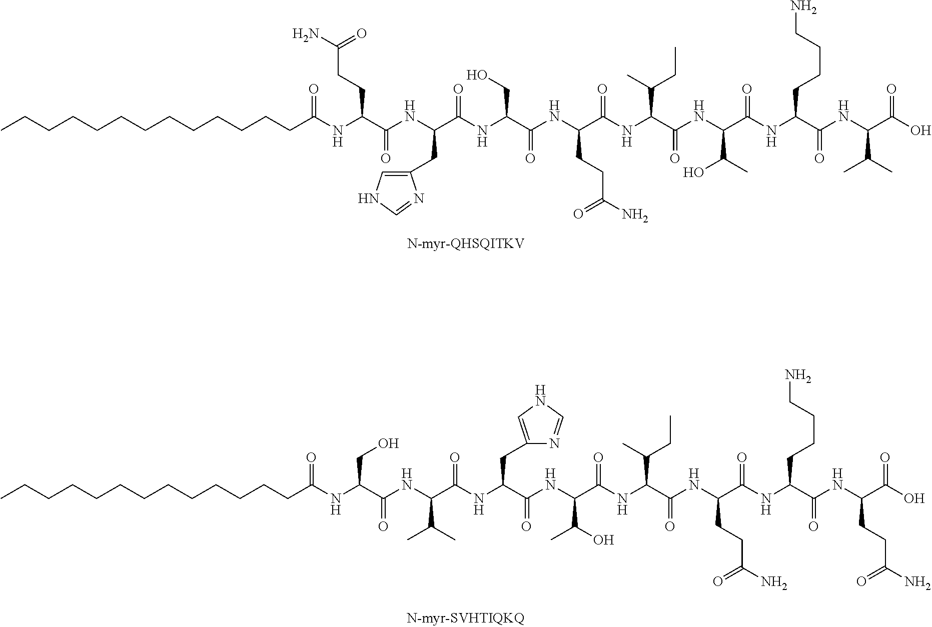 [SMILES: CCCCCCCCCCCCCC(=O)N[C@@H](CCC(N)=O)C(=O)N[C@H](Cc1c[nH]cn1)C(=O)N[C@@H](CO)C(=O)N[C@H](CCC(N)=O)C(=O)N[C@H](C(=O)N[C@@H](C(=O)N[C@@H](CCCCN)C(=O)N[C@@H](C(=O)O)C(C)C)C(C)O)C(C)CC ; CCCCCCCCCCCCCC(=O)N[C@@H](CO)C(=O)N[C@@H](C(=O)N[C@@H](Cc1c[nH]cn1)C(=O)N[C@@H](C(=O)N[C@H](C(=O)N[C@H](CCC(N)=O)C(=O)N[C@@H](CCCCN)C(=O)N[C@H](CCC(N)=O)C(=O)O)C(C)CC)C(C)O)C(C)C]

Time lapse imaging of the fluorescein-tagged peptides revealed their rapid (in seconds) diffusion into neurons, including dendritic spines. It was posited that the PTEN-PDZ peptide would saturate PDZ interaction sites of PTEN, consequently preventing anchoring of PTEN to PDZ proteins, and in this manner it would prevent LTD. Indeed, and in a striking similarity to PTEN inhibition with drugs or dominant negative mutants (Jurado, et al. (2010) EMBO J. 29:2827-2840), incubation of acute mice slices in PTEN-PDZ peptide (10 μM, 1-2.5 hours) completely blocked LTD, whereas the scrambled peptide did not have any effect (FIG. 4).

The effect of these peptides on Aβ-treated neurons was subsequently tested. Preincubation of slices with the PTEN-PDZ peptide for 1 hour prior to the addition of synthetic protofibrillar Aβ prevented synaptic depression, whereas the control-scrambled peptide did not (neither peptide altered basal synaptic transmission in the absence of Aβ treatment). These results demonstrate that a pharmacological tool to saturate PTEN-PDZ interaction sites effectively protects synapses against Aβ.

To test the in vivo effects of this peptide, the PTEN-PDZ peptide was infused into cerebral ventricles of APP/PS1 transgenic mice (n=5). The peptide was chronically administrated (3-4 weeks infusion time) through a cannula connected to an Alzet miniosmotic pump with a concentration of the peptide in the pump of 2 mM. APP/PS1 transgenic mice (n=6) treated with vehicle and wild-type mice (n=4) treated with the PTEN-PDZ peptide were used as controls. Animals were subjected to the object location memory test described herein. Upon intracerebroventricular injection of this peptide, APP/PS1 mice showed a significant improvement in a spatial learning task (p=0.027, compared to vehicle-infused APP/PS1 mice), to the extent that their recognition index was similar to that of wild-type mice (FIG. 5).

What is claimed is:

1. A method for mitigating or alleviating synaptic and cognitive deficits associated with a β-amyloidogenic disease comprising administering an effective amount of a peptide of SEQ ID NO:3, or a derivative or peptidomimetic thereof, to a subject in need of treatment so that synaptic and cognitive deficits associated with a β-amyloidogenic disease are mitigated or alleviated.

2. The method of claim 1, wherein the β-amyloidogenic disease is Alzheimer's disease.

3. The method of claim 1, wherein the peptide, derivative or peptidomimetic is administered to the subject by direct injection.

4. The method of claim 3, wherein the peptide, derivative or peptidomimetic is directly injected into the brain of the subject.

5. The method of claim 1, wherein the derivative is N-myristoyl-QHSQITKV (SEQ ID NO:2) or N-myristoyl-QHTQITKV (SEQ ID NO:25).

6. A kit comprising an effective amount of a peptide derivative comprising N-myristoyl-QHSQITKV (SEQ ID NO:2) or N-myristoyl-QHTQITKV (SEQ ID NO:25); and a pharmaceutically acceptable carrier.
